(19) United States
(12) Patent Application Publication
(10) Pub. No.: US 2013/0065820 A1
(43) Pub. Date: Mar. 14, 2013

(54) SYNTHETIC MYOSTATIN PEPTIDE ANTAGONISTS

(71) Applicants: Robert Syndecombe Bower, Peregian Beach (AU); Monica Senna Salerno De Moura, Hamilton (NZ); Gina Diane Nicholas, Hamilton (NZ); Mark Francis Thomas, Hamilton (NZ); Carole Judith Berry, Hamilton (NZ)

(72) Inventors: Robert Syndecombe Bower, Peregian Beach (AU); Monica Senna Salerno De Moura, Hamilton (NZ); Gina Diane Nicholas, Hamilton (NZ); Mark Francis Thomas, Hamilton (NZ); Carole Judith Berry, Hamilton (NZ)

(73) Assignee: COVITA LIMITED, Hamilton (NZ)

(21) Appl. No.: 13/499,566

(22) PCT Filed: Sep. 29, 2010
(86) PCT No.: PCT/NZ10/00191

§ 371 Date: Nov. 9, 2012

Related U.S. Application Data
(60) Provisional application No. 61/247,821, filed on Oct. 1, 2009.

(51) Int. Cl. A61K 38/18; C07K 1/06; A61P 21/00; A61P 17/02; A61P 3/10; A61P 19/10; A61P 1/16; A61P 13/12; A61P 3/04; C07K 14/495; A61P 9/04

(52) U.S. Cl. 514/4.8; 530/324; 514/8.9; 514/6.9

(57) ABSTRACT

The present invention relates to novel synthetic myostatin antagonists, comprising a synthetic mature myostatin peptide, wherein the peptide comprises at least two cysteine residues at positions 281 and 282 which are forced to bond and form a disulfide bond or a functional variant or fragment thereof and are useful in the treatment of myostatin related disorders.

### FIELD OF THE INVENTION

The present invention relates to synthetic peptides having myostatin antagonist activity and their use in the treatment of myostatin related disorders.

### BACKGROUND

Myostatin antagonist peptides are known in the art and are usually produced by recombinant techniques. However, recombinant methods often include the use of tag sequences which can be difficult to remove and therefore remain on the biologically active molecule. It is not known if these tag sequences interfere with the biological activity. In addition, recombinant techniques are usually carried out in bacteria, such as E. coli, which may result in bacterial post translational modifications that affect the biological activity of the recombinant molecule in a mammalian system. Bacterially produced recombinant peptides can also vary in their three dimensional structure as it is not possible to control the formation of cysteine-cysteine disulfide bonds in such bacterial systems. This leads to inconsistencies in the production of the same recombinantly produced peptide with resultant variation in biological activity.

For these reasons it would be desirable to use synthetic methods for synthesizing myostatin antagonists to ensure the production of “clean” peptides with no tag sequences or bacterial post-translational modifications. However, other problems arise with the use of peptide synthesizers. Peptides with multiple cysteine, methionine, arginine and tryptophan residues are difficult to synthesize and even if they can be successfully synthesized, are often insoluble and therefore impossible to use in vivo. Beside synthesis problems, there are also other post-synthesis adduct formation and modification problems associated with peptides.

Synthesized peptides are usually lyophilised, and stored as a dry powder at −20° C. to −70° C. Even in these conditions, peptides can degrade, especially cysteine containing peptides.

In addition, peptides that have multiple cysteine residues will have multiple connectivities giving rise to a mixture of biologically active and non-active peptides. As the production of synthetic peptides is expensive and often only very small amounts are synthesized, a synthesized peptide comprising a mixture of connectivities, may not comprise a sufficient amount of the active form to be biologically active.

Myostatin peptides comprise multiple cysteine residues and so present numerous difficulties in synthesizing biologically active peptides. However, as mentioned above, recombinant peptides are not ideal as the presence of tag sequences and/or microbial post-translational modifications may present regulatory hurdles to the development of myostatin antagonists for clinical use, and the problems in obtaining the correct three dimensional structure means that the recombinantly produced peptide may not be active or fully active. A need therefore exists for “clean” synthetic peptides that are biologically active.

Accordingly, it is an object of the present invention to provide synthetic peptides having antagonist activity for the treatment of myostatin related disorders, and/or to provide the public with a useful choice.

### SUMMARY OF THE INVENTION

The present invention is directed to novel synthetic peptides having myostatin antagonist activity.

In a first embodiment, the present invention provides a synthetic peptide having myostatin antagonist activity having an amino acid sequence corresponding to at least five contiguous amino acids of a mature myostatin peptide of SEQ ID NO:1, wherein the synthetic peptide comprises at least two cysteine residues at positions 281 and 282 which are forced to bond and form a disulfide bond, or a functional variant or fragment thereof.

Preferably, the synthetic peptide of the invention comprises at least ten, at least fifteen, at least twenty, at least twenty five, at least thirty, at least thirty five, at least forty, at least forty five, at least fifty, at least fifty five, or at least sixty contiguous amino acids of SEQ ID NO:1.

Preferably, the synthetic peptide of the invention comprises an amino acid sequence corresponding to a C-terminally truncated mature myostatin peptide of SEQ ID NO: 1, wherein the C-terminal truncation is at or between amino acids 282 and 335 said peptide comprising at least two cysteine residues at positions 281 and 282 which are forced to bond and form a disulfide bond; or a functional variant or fragment thereof.

The myostatin amino acid sequence of SEQ ID NO: 1 comprises the precursor myostatin protein having 375 amino acids. The active mature C-terminal myostatin protein is cleaved at Arg 266 by the action of furin endoprotease.

The preferred active synthetic peptide of the present invention therefore comprises an amino acid sequence corresponding to at least five contiguous amino acids from position 267 to the C-terminal truncation position, of SEQ ID NO:1. Optionally, the peptide may include a N-terminal truncation at or between position 268 to 280.

The cysteine residue at amino acid positions 272, 281, 282 and 309 of SEQ ID NO: 1 are cysteines 1, 2, 3 and 4 respectively. Additional cysteine residues present in the synthetic peptide are numbered sequentially as would be understood by a skilled worker.

The present invention is directed to synthetic peptides having at least two cysteine residues bonded to form a disulfide bond, wherein the disulfide bond connectivity is between cysteines 2 and 3. This connectivity is not thought to be a natural connectivity and these two cysteine residues are forced to bond during the synthesis process.

The synthetic peptide may be selected from the group consisting of an amino acid sequence corresponding to a C-terminally truncated mature myostatin peptide wherein the C-terminal truncation is at amino acid position 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334 or 335 of SEQ ID NO:1, or functional variant or fragment thereof.

Preferably the synthetic peptide of the invention is selected from the group consisting of an amino acid sequence corresponding to a C-terminally truncated mature myostatin peptide, wherein the C-terminal truncation is at amino acid position 329, 320, 310, 300, 295, 289, 284 or 282 (SEQ ID NOS: 2-9), or a functional variant or fragment thereof, or a peptide having substantial sequence homology thereto.

More preferably the synthetic peptide of the invention is selected from the group consisting of an amino acid sequence corresponding to a C-terminally truncated mature myostatin peptide, wherein the C-terminal truncation is at amino acid position 320, 310 or 300 (SEQ ID NOS: 3-5) or a functional variant or fragment thereof, or a peptide having substantial sequence homology thereto.

Most preferably, the invention is directed to a synthetic 310 truncated peptide (SEQ ID NO:4). This peptide comprises four cysteine residues (cysteines 1, 2, 3 and 4 respectively). As the peptide of the invention has a forced 2-3 connectivity, this preferred synthetic peptide can comprise 2-3 connectivity only (when the 1 and 4 cysteine residues remain protected) or can comprise a mixture of 2-3/1-4 connectivities.

of the synthetic peptide sequences of the invention may be desirable as a way to produce a myostatin antagonist having selectively altered binding characteristics or having improved biodistribution or half life in vivo or on the shelf, or improved solubility, including glycosylation, PEGylation, farnesylation, acetylation, biotinylation, D amino acid substitution, or organic derivatization, as would be understood by a skilled worker.

The present invention also provides a method of synthesizing peptides having myostatin antagonist activity, having an amino acid sequence corresponding to at least five contiguous amino acids of a mature myostatin peptide of SEQ ID NO: 1, wherein the cysteine residues are protected and deprotected to force a disulfide bond connectivity between cysteines 2 and 3 (at positions 281 and 282), or between cysteines 2 and 3 and between cysteines 1 and 4 (at positions 281 and 282 and at positions 272 and 309).

The invention also provides for a pharmaceutical composition comprising at least one synthetic peptide of the invention together with a pharmaceutically acceptable carrier.

The present invention also provides a method of regulating muscle growth, promoting adipogenic differentiation and/or promoting bone growth or mineralization in an animal comprising administering to said animal an effective amount of at least one synthetic peptide of the invention. Preferably, the method may be used to increase muscle mass, decrease fat deposition and/or, improve bone growth in a sheep, cattle, deer, poultry, turkey, pig, horse, mouse, rat, cat, dog or human.

The animal may have normal or abnormal levels of myostatin. In animals with normal levels of myostatin, treatment with the antagonists of the invention will result in increased muscle mass. In animals with normal muscle mass, such treatment will result in an increase in muscle mass and may be particularly useful in the meat production industry. In animals with reduced muscle mass, due to muscle damage or trauma, wasting due to bed rest, etc, treatment with the antagonists of the invention will restore the muscle mass to normal. In animals with abnormal myostatin levels, the muscle mass will invariably be reduced and treatment with myostatin antagonists of the invention will restore the muscle mass back towards normal levels.

The invention also provides a method to prevent, treat or reduce the severity of a myostatin related pathologic condition in a patient, wherein the condition is characterized, at least in part, by an abnormal amount, development or metabolic activity of muscle or adipose tissue, wherein said method comprises administering an effective amount of at least one synthetic peptide of the invention to a patient in need thereof.

The pathologic condition may include disorders related to muscle hypertrophy; muscle atrophy and muscle wasting associated with inflammatory myopathies, muscular dystrophies, motor neuron diseases, diseases of the neuromuscular junction, diseases of the peripheral nerve, myopathies due to endocrine abnormalities, metabolic syndrome, HIV, cancer, sarcopenia, cachexia, inactivity or prolonged bedrest and other wasting conditions; cardiac failure; osteoporosis; renal failure or disease; liver failure or disease; anorexia; obesity; diabetes; and wound healing.

As another alternative a synthetic peptide of this invention may be conjugated to another pharmaceutically active compound to enhance the therapeutic effect on the target cell or tissue by delivering a second compound in an effort to treat the diseases or therapeutic indications stated above. In these combinations, the myostatin antagonist of the invention may be independently and sequentially administered or co-administered.

The present invention also provides a method of regulating muscle growth of an animal comprising administering to said animal an effective amount of at least one synthetic peptide of the invention. Preferably, the method may be used to produce increased muscle mass in a sheep, cattle, deer, poultry, turkey, pig, horse, mouse, rat, cat, dog or human.

### EXEMPLARY DEFINITIONS

Unless defined otherwise, all technical and scientific terms used herein have the same meaning as commonly understood by one of ordinary skill in the art to which this invention belongs. Although any methods and compositions similar or equivalent to those described herein can be used in the practice or testing of the present invention, the preferred methods and compositions are described herein. For purposes of the present invention, the following terms are defined below:

“Hypertrophy” as used throughout the specification and claims means any increase in cell size.

“Hyperplasia” as used throughout the specification and claims mean any increase in cell number.

“Muscle atrophy” as used throughout the specification and claims means any wasting or loss of muscle tissue resulting from the lack of use.

“Sarcopenia” as used throughout the specification and claims means a decline in muscle mass and performance caused by old age, as well as sarcopenia-related or other age-related muscle disorders characterised by muscle atrophy and a decrease in the ability of satellite cells to become activated.

“Inhibitor” or “antagonist” of myostatin as used throughout the specification and claims means any compound that acts to decrease, either in whole or in part, the activity of myostatin.

“Muscle growth” is to be understood as meaning the division and/or differentiation of muscle cells and includes the division and/or differentiation of any precursor cell, fusion of such cells with each other and/or with existing muscle fibres, and it also includes increased protein synthesis in myofibres leading to higher protein content and greater muscle fibre volume (muscle fibre hypertrophy).

A “peptide” or “polypeptide” as used herein is to be understood as meaning a synthetically made polymer of naturally occurring and/or artificial amino acids covalently linked via peptide bonds. Polypeptides that have been isolated from a naturally occurring source, or produced using recombinant techniques are not included.

The term “fragment or variant” is to be understood to mean any peptide sequence or partial sequence that has been modified by substitution, insertion or deletion of one or more amino acids, but that has substantially the same activity or function as the unmodified sequence or partial sequence.

Preferably, a variant contains conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine, and valine; glycine, and alanine; asparagine and glutamine; and serine, threonine, phenylalanine, and tyrosine. Other groups of amino acids that may represent conservative changes include (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his.

Amino acids may be classified according to the nature of their side groups. Amino acids with nonpolar alkyl side groups include glycine, alanine, valine, leucine, and isoleucine. Serine and threonine have hydroxyl groups on their side chains, and because hydroxyl groups are polar and capable of hydrogen bonding, these amino acids are hydrophilic. Sulfur groups may be found in methionine and cysteine. Carboxylic acid groups are part of the side chain of aspartic acid and glutamic acid, which because of the acidity of the carboxylic acid group, the amino acids are not only polar but can become negatively charged in solution. Glutamine and asparagine are similar to glutamic acid and aspartic acid except the side chains contain amide groups. Lysine, arginine, and histidine have one or more amino groups in their side chains, which can accept protons, and thus these amino acids act as bases. Aromatic groups may be found on the side chains of phenylalanine, tyrosine, and tryptophan. Tyrosine is polar because of its hydroxyl group, but tryptophan and phenylalanine are non-polar. A variant may also, or alternatively, contain nonconservative changes.

A synthetic peptide of the invention, or a functional variant or fragment thereof has the biological function of antagonising myostatin activity. To determine whether a synthetic peptide of the invention, or functional variant or fragment thereof, is able to antagonise myostatin activity, such activity can be tested by growing myoblasts in the presence or absence (control) of a candidate synthetic peptide of the invention. An increase in the growth of myoblasts, which produce myostatin and therefore limit their own rate of proliferation, over control myoblasts that did not receive the candidate peptide indicates that the peptide has myostatin antagonistic activity. A suitable cell line could be murine C₂Cl₂ myoblasts (ATCC NO: CRL-1772), however, it will be appreciated that any suitable myoblast cell line could be used, such as primary ovine, bovine, porcine or human myoblasts.

The term ‘comprising’ as used in this specification and claims means ‘consisting at least in part of”, that is to say when interpreting independent claims including that term, the features prefaced by that term in each claim all need to be present but other features can also be present.

The terms “substantially corresponds to,” “substantially homologous,” or “substantial identity” as used herein denotes a characteristic of an amino acid sequence, wherein a selected amino acid sequence has at least about 70 or about 75 percent sequence identity as compared to a selected reference amino acid sequence. More typically, the selected sequence and the reference sequence will have at least about 76, 77, 78, 79, 80, 81, 82, 83, 84 or even 85 percent sequence identity, and more preferably at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared. The percentage of sequence identity may be calculated over the entire length of the sequences to be compared, or may be calculated by excluding small deletions or additions which total less than about 25 percent or so of the chosen reference sequence, for example using sequence comparison algorithms well-known to those of skill in the art, such as, the FASTA program analysis described by Pearson and Lipman (1988) and the gapped BLAST algorithm (e.g., Altschul et al.) which weights sequence gaps and sequence mismatches according to the default weightings provided by the National Institutes of Health/NCBI database (Bethesda, Md.; see Internet:>www.ncbi.nlm.nih.gov/cgi-bin/BLAST/nph-newblast).

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

### DETAILED DESCRIPTION OF THE INVENTION

The present invention is directed to novel synthetic peptides possessing myostatin antagonist activity for use in the treatment of myostatin related disorders.

Specifically, the present invention provides a synthetic peptide having myostatin antagonist activity having an amino acid sequence corresponding to at least five contiguous amino acids of a mature myostatin peptide of SEQ ID NO:1, wherein the synthetic peptide comprises at least two cysteine residues at positions 281 and 282 which are forced to bond and form a disulfide bond, or a functional variant or fragment thereof.

Preferably, the synthetic peptide of the invention comprises at least ten, at least fifteen, at least twenty, at least twenty five, at least thirty, at least thirty five, at least forty, at least forty five, at least fifty, at least fifty five, or at least sixty contiguous amino acids of SEQ ID NO:1.

Preferably the invention is directed to novel synthetic dominant negatives of myostatin comprising an amino acid sequence corresponding to a mature myostatin peptide having a C-terminal truncation at a position either at or between amino acid positions 282 to 335 of SEQ ID NO: 1, said peptide comprising at least two cysteine residues at positions 281 and 282, which are forced to bond and form a disulfide bond, or a functional variant or fragment thereof.

The myostatin protein is initially translated as a 375 amino acid precursor molecule having α-secretory signal sequence at the N-terminus, a proteolytic processing signal (RSRR) of the furin endoprotease, and nine conserved cysteine residues in the C-terminal region to facilitate the formation of a “cysteine knot” structure. Myostatin is activated by furin endoprotease cleavage at Arg 266 releasing the N-terminal, or “latency-associated peptide” (LAP) and the mature, C-terminal domain, which dimerises to form the active myostatin molecule. After processing, a homodimer of the LAP peptide remains non-covalently bound to the homodimer of mature myostatin in an inactive complex (Lee et al, 2001). The amino acid sequence of the human myostatin precursor protein molecule is shown in SEQ ID NO: 1.

Myostatin antagonists comprising mature myostatin peptides which are C-terminally truncated at amino acid position 330 or 350 are known (WO 2001/53350). These antagonists are truncated at a position so that key cysteines are retained that are likely to play an important role in determining their three dimensional structure and associated interactions with other molecules. Loss of these key cysteine residues would be expected to negatively impact on their ability to function (Jeanplong et al, 2001).

However, mature recombinant myostatin peptides C-terminally truncated at positions close to or excluding cysteine residues have been shown to be biologically active (WO 2008/016314). WO 2008/016314 teaches that at least two cysteine moieties are required in a recombinant C-truncated mature myostatin peptide in order to retain biological activity although the exact conformational form of the biologically active peptides are not known. WO 2008/016314 further contends that recombinantly produced peptides are necessary to retain biological activity as synthetic versions could not be produced that had any biological activity.

Surprisingly, it has been shown for the first time that biologically active C-terminally truncated mature myostatin peptides can be synthesized.

In addition, it has been shown for the first time that synthetic myostatin peptides are required to be synthesized so that cysteine residues at positions 281 and 282 of SEQ ID NO: 1 are forced to bond and form a disulfide bond in order to obtain biological activity. These cysteine residues are the second and third cysteines in the peptide sequences of the invention and will be referred to as the 2-3 connectivity. As these cysteine residues are side by side in the amino, acid sequence it is unlikely that they would naturally bond together and the cysteine residues in the synthetic peptide of the invention must be protected and deprotected to force the 2-3 connectivity. As this connectivity is therefore unlikely to occur in a native or recombinant myostatin peptide, this result was highly surprising.

The synthetic peptides of the present invention comprise at least five contiguous amino acids of an amino acid sequence corresponding to the sequence consisting of amino acids 267 (cleavage site) to 335 of SEQ ID NO: 1, includes cysteine residues at positions 281 and 282 as follows:

```
                                          1 2 3
Asp Phe Gly Leu Asp Cys Asp Glu His Ser Thr Glu Ser Arg Cys Cys Arg Tyr Pro
                        270 275 280 285
Leu Thr Val Asp Phe Glu Ala Phe Gly Trp Asp Trp Ile Ile Ala Pro Lys Arg Tyr
                                290 295 300
                                  4 5
Lys Ala Asn Tyr Cys Ser Gly Glu Cys Glu Phe Val Phe Leu Gln Lys
305 310 315 320
Tyr Pro His Thr His Leu Val His Gln Ala Asn Pro Arg Gly Ser
                                325 330 335
```

The synthetic peptides of the invention can comprise between two and five cysteine residues depending on the truncation position (from between amino acid position 282 and 335). The cysteine residues bond to form disulfide bonds. There are many alternative disulfide connectivities that are possible depending on how many cysteine residues are present in a synthetic peptide of the invention.

When the truncation position is at or between amino acids 282 to 308, the synthetic peptide comprises three cysteine residues so that possible cysteine connectivities include 1-2, 1-3 and 2-3.

When the truncation position is at or between amino acids 309 to 312, the synthetic peptide comprises four cysteine residues so that possible cysteine connectivities include 1-2, 1-3, 1-4, 2-3, 2-4 and 3-4.

When the truncation position is at or between amino acids 313 to 335, the synthetic peptide comprises five cysteine residues so that possible cysteine connectivities include 1-2, 1-3, 1-4, 1-5; 2-3, 2-4, 2-5, 3-4, 3-5 and 4-5.

It is likely that a native myostatin C-terminal truncated peptide would include a mixture of all possible connectivities. As a skilled worker is aware, the correct peptide connectivity is crucial for biological activity as it determines the three dimensional structure required for biological activity.

From the teaching of WO 2008/016314, it was thought that the 1-2 connectivity would be required for biological activity as this disclosure showed that a mature recombinant myostatin peptide C-terminally truncated at position 281 was biologically active. This recombinant peptide only had two cysteine residue and could only form a 1-2 connectivity, so that this connectivity appeared to be crucial to myostatin bioactivity.

Surprisingly, the present inventors found that a synthetic peptide having a truncation position at 281 was not biologically active. In addition the present inventors found that at least two cysteine residues are required for biological activity of a synthetic peptide and further that the 2-3 connectivity was the crucial connectivity for bioactivity of the synthetic peptides of the invention. This was unexpected from the teaching of WO 2008/016314.

The present inventors tested all other possible connectivities and found that the 1-2, 1-3, 1-4, 2-4, 3-4 and various mixtures thereof were not biologically active. However, a synthetic peptide corresponding to a 310 truncation and comprising a mixture of 2-3/1-4 connectivities was biologically active. This was considered to be due mainly to the presence of the 2-3 peptide.

The present invention therefore provides a synthetic peptide having myostatin antagonist activity having an amino acid sequence corresponding to at least five contiguous amino acids of a mature myostatin peptide of SEQ ID NO:1, wherein the synthetic peptide comprises at least two cysteine residues at positions 281 and 282 which are forced to bond and form a disulfide bond, or a functional variant or fragment thereof.

Preferably, the synthetic peptide of the invention comprises at least ten, at least fifteen, at least twenty, at least twenty five, at least thirty, at least thirty five, at least forty, at least forty five, at least fifty, at least fifty five, or at least sixty contiguous amino acids of SEQ ID NO:1.

Preferably the invention provides a synthetic peptide having myostatin antagonist activity, comprising an amino acid sequence corresponding to a C-terminally truncated mature myostatin peptide of SEQ ID NO:1, wherein the C-terminal truncation is at a position at or between amino acids 282 and 335, said peptide comprising at least two cysteine residues at positions 281 and 282 which are forced to bond and form a disulfide bond, or a functional variant or fragment thereof.

The peptides of the invention thus comprise a 2-3 connectivity. The synthetic peptides of the invention may also comprise a mixture of a 2-3/1-4 connectivities.

The synthetic peptides of the invention may be selected from the group consisting of an amino acid sequence corresponding to a C-terminally truncated mature myostatin peptide wherein the C-terminal truncation is at amino acid position 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334 or 335 of SEQ ID NO: 1, or a functional variant or fragment thereof.

Preferably the synthetic peptide of the invention is selected from the group consisting of an amino acid sequence corresponding to a C-terminally truncated mature myostatin polypeptide, wherein the C-terminal truncation is at amino acid position 329, 320, 310, 300, 295, 289, 284 or 282 (SEQ ID NOS: 2-9), or a functional variant or fragment thereof, or a polypeptide having substantial sequence homology thereto.

More preferably the synthetic peptide of the invention is selected from the group consisting of an amino acid sequence corresponding to a C-terminally truncated mature myostatin polypeptide, where in the C-terminal truncation is at amino acid position 320, 310 or 300 (SEQ ID NOs: 3, 4 or 5).

The synthetic peptides of this invention can be altered in many ways to produce variants having improved pharmacokinetics, as would be appreciated by a skilled worker. For example, functional groups may be added that alter polarity and/or the ability to form hydrogen bonds and will alter the solubility of the synthetic peptides. Similarly a functional group may alter the stability by changing the serum half-life (Werle et al, 2006) or by controlling the release of the synthetic peptide from a micelle at the target site. Further a functional group may alter biocompatibility, for example by minimizing the side effects of the synthetic peptide to the patient. Addition of a functional group-capable of binding to target cells or tissues or facilitating the transport into the target cells will enhance delivery and targeting of the synthetic peptide.

A functional group conjugated to a synthetic peptide of this invention may be a biological targeting molecule that binds to a specific biological substance or site. The biological substance or site is the intended target of the delivery and targeting molecule that binds to it, enabling the delivery of the synthetic peptide to the tissue or cells of interest.

A ligand may function as a biological targeting molecule by selectively binding or having a specific affinity for another substance. A ligand is recognized and bound by a specific binding body or binding partner, or receptor. Examples of ligands suitable for targeting are antigens, haptens, biotin, biotin derivatives, lectins, galactosamine and fucosylamine moieties, receptors, substrates, coenzymes and cofactors among others. Other substances that can function as ligands for delivery and targeting are certain steroids, prostaglandins, carbohydrates, lipids, certain proteins or protein fragments (i.e. hormones, toxins), and synthetic or natural polypeptides with cell affinity. Ligands also include various substances with selective affinity for ligators that are produced through recombinant DNA, genetic and molecular engineering.

Another type of targeting molecule is an antibody, which term is used herein to include all classes of antibodies, monoclonal antibodies, chimeric antibodies, Fab fractions, fragments and derivatives thereof. Other targeting molecules include enzymes, especially cell surface enzymes such as neuraminidases, plasma proteins, avidins, streptavidins, chalones, cavitands, thyroglobulin, intrinsic factor, globulins, chelators, surfactants, organometallic substances, staphylococcal protein A, protein G, cytochromes, lectins, certain resins, and organic polymers. Targeting molecules may include peptides, including proteins, protein fragments or polypeptides which may be produced synthetically or through recombinant techniques known in the art. Examples of peptides include membrane transfer proteins which could facilitate the transfer of the compound to a target cell interior or for nuclear translocation (see: WO 01/15511).

Other modifications to the synthetic peptides of the invention include conjugates to a biologically compatible polymer such as polyethylene glycol (PEG) and related polymer derivatives. Drug-PEG conjugates have been described as improving the circulation time (prolong serum half-life) before hydrolytic breakdown of the conjugate and subsequent release of the bound molecule thus increasing the drugs efficacy. For example, U.S. Pat. No. 6,214,966 describes the use of PEG and related polymer derivatives to conjugate to drugs such as proteins, enzymes and small molecules to improve the solubility and to facilitate controlled release of the drug. Alternatively, EP 1082105 (WO 99/59548) describes the use of biodegradable polyester polymers as a drug delivery system to facilitate controlled release of the conjugated drug.

As another alternative a synthetic peptide of this invention may be conjugated to another pharmaceutically active compound to enhance the therapeutic effect on the target cell or tissue by delivering a second compound with a similar myostatin antagonistic effect or a different activity altogether. For example, U.S. Pat. No. 6,051,576 describes the use of co-drug formulations by conjugating two or more agents via a labile linkage to improve the pharmaceutical and pharmacological properties of pharmacologically active compounds. For example, a second myostatin antagonist may be selected from any one or more known myostatin inhibitors. For example, U.S. Pat. No. 6,096,506 and U.S. Pat. No. 6,468,535 disclose anti-myostatin antibodies. U.S. Pat. No. 6,369,201 and WO 01/05820 teach myostatin peptide immunogens, myostatin multimers and myostatin immunoconjugates capable of eliciting an immune response and blocking myostatin activity. Protein inhibitors of myostatin are disclosed in WO 02/085306, which include the truncated Activin type II receptor, the myostatin pro-domain, and follistatin. Other myostatin inhibitors derived from the myostatin peptide are known, and include for example myostatin inhibitors that are released into culture from cells overexpressing myostatin (WO 00/43781); dominant negatives of myostatin (WO 01/53350), which include the Piedmontese allele (cysteine at position 313 is replaced with a tyrosine) and mature myostatin peptides having a C-terminal truncation at a position either at or between amino acid positions 330 to 375. US2004/0181033 also teaches small peptides comprising the amino acid sequence WMCPP, and which are capable of binding to and inhibiting myostatin.

A second pharmacologically active compound having different activity to the synthetic myostatin antagonist peptide of the invention may be used conjointly with the synthetic peptide of the invention to treat the myostatin related disorders. For example, the synthetic peptide may be administered in conjunction with polypeptide growth factors, NSAIDs or COX-2 inhibitors, alpha and beta blockers, ACE inhibitors, bisphosphonates, oestrogen receptor modulators, antihypertensive agents, glutamate antagonists, insulin, antibiotics, protein kinase C inhibitors or various over the counter substances as would be appreciated by a skilled worker.

Other modifications to improve stability and half life include the identification of susceptible amino acid protease cleavage sites within the synthetic peptides of the invention, and replacement of such amino acids with alternative amino acids to prevent protease degradation of the synthetic peptide in plasma, in vivo. A person skilled in the art will appreciate what type of functional groups might be added to achieve the desired result in administering the synthetic peptide to the patient and thereby improving the overall therapeutic index.

The specific synthetically produced C-terminally-truncated polypeptides exemplified in the present invention are shown in relation to their position on the C-terminal portion of myostatin of SEQ ID NO: 1.

Also included are synthetic peptides whose sequence differs from those of the invention by one or more conservative amino acid substitutions, deletions or insertions which do not affect the biological activity of the synthetic peptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Examples of conservative substitutions can be taken from Table 1 below.

TABLE 1
CONSERVATIVE AMINO ACID REPLACEMENTS

| For Amino Acid | Code | Replace with any of |
|---|---|---|
| Alanine | A | D-Ala, Gly, beta-Ala, L-Cvs, D-Cys |
| Arginine | R | D-Arg, Lys, D-Lvs, homo-Arg, D-homo-Arg, |
| | | Met, Ile, D-Met-, D-Ile,Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys; Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, .beta.-Ala Acp |
| Histidine | H | Asp, D-Asp, Lys, D-Lys, Tyr |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, D-Val, |
| | | D-Val |
| Phenylalanine | F | D-Phe, Tyr, D-Thr, L-Dopa, His, D-His, Trp, |
| | | D-Trp, Trans-3,4, or 5-phenylproline, cis-3,4, or 5- |
| | | phenylproline |
| Proline | P | D-Pro, L-I-thioazolidine-4-carboxylic acid, D- |
| | | or L-1-oxazolidine4-carboxylic acid |
| Serine | S | D-Ser, Thr, D-Thr, allo-Thr, Met, D-Met, |
| | | Met(O); D-Met(O), L-Cys, D-Cys |
| Threonine | T | D-Thr, Ser, D-Ser, allo-Thr, Met; D-Met, Met(O), |
| | | D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Other variants include synthetic peptides with modifications which influence peptide stability. Such variants may contain, for example, one or more non-peptide bonds (which replace the peptide bonds) in the synthetic peptide sequence. Also included are variants that include residues other than naturally occurring L-amino acids, e.g. D-amino acids or non-naturally occurring synthetic amino acids, e.g. beta or gamma amino acids and cyclic variants.

The present invention further contemplates synthetic peptides of the invention further comprising N-terminal truncations of the peptide, whereby the amino acids removed at the N-terminal end are not synthesized and whereby such synthetic peptides retain myostatin antagonistic activity. As described above, such modified peptides will include at least two cysteine residues, including the cysteine residues at positions 281 and 282 of SEQ ID. NO: 1.

Recent studies suggest that myostatin is a potent regulator of cell cycle progression and functions in part by regulating both the proliferation and differentiation steps of myogenesis (Langley et al., 2004; Thomas et al., 2000). Several studies have demonstrated a role for myostatin not only during embryonic myogenesis, but also in postnatal muscle growth. Studies by Wehling et al (Wehling et al., 2000) and Carlson et al (Carlson et al., 1999) indicated that atrophy-related muscle loss due to hind limb suspension in mice was associated with increased myostatin levels in M. plantaris. Increased myostatin levels were also associated with severe muscle wasting seen in HIV patients (Gonzalez-Cadavid et al., 1998). One explanation for the elevated levels of myostatin observed during muscle disuse is that myostatin may function as an inhibitor of satellite cell activation. Indeed this is supported by recent studies which show that a lack of myostatin results in an increased pool of activated satellite cells in vivo and enhanced self-renewal of satellite cells (McCroskery et al., 2003). Myostatin inhibitors have also been shown to increase the activation of satellite cells, as well as to increase the migration of macrophages and myoblasts during muscle regeneration (WO02006/083182) and in wound healing (WO2006/083183).

Methods suitable for assaying for myostatin antagonist activity of the synthetic peptides of the present invention may be based on any of a variety of known methodologies including known in vivo animal models or in vitro models. For example, potential myostatin antagonists of the invention may first be tested using an in vitro single fibre satellite cell activation assays, myoblasts proliferation assays, bioassay (WO 2003/00120) or myoblast and/or macrophage migration assays, as described in WO 2008/016314. Myostatin antagonist synthetic peptides of the invention that are able to increase satellite cell activation, myoblast proliferation and/or myoblast or macrophage migration in vitro, may then be tested for their ability to treat myostatin related disorders in animal models in vivo. Such models include an aged mouse model of sarcopenia (Kirk, 2000); a mouse model of muscular dystrophy (Mdx) (Tanabe et al, 1986); a mouse model of diabetes (Like et al, 1976); a mouse model of obesity (Giridharan et al, 1998); a notexin model of muscle injury (Kirk, 2000); a model of superficial or deep skin wounds (Shukla et al, 1998); a model of burns (Yang et al, 2005); a mouse cachexia model where dexamethasone is injected into mice to induce muscle wasting (Ma et al, 2003) or a mouse cancer model in which colon adenocarcinoma (C26) cells or Lewis Lung carcinoma (LLC) cells are injected into mice to induce muscle wasting associated with cancer.

The synthetic peptides of the invention preferably bind to their target with a Kd of 1 μM or less, and more preferably with a Kd of 100 nM, 10 nM or even 1 nM or less.

The present invention is also directed to a pharmaceutical composition comprising at least one novel synthetic peptide of the invention having myostatin antagonistic activity together with a pharmaceutical or physiologically acceptable carrier.

A synthetic peptide of this invention or salt thereof may be included in a pharmaceutically acceptable carrier or diluent, ideally in an amount sufficient to deliver to a patient a therapeutically effective amount without causing serious toxic effects in the patient treated. The concentration of active synthetic peptide in the composition will depend on absorption, distribution, inactivation, and excretion rates of the synthetic peptide as well as other factors known to those of skill in the art. It is to be noted that dosage values will also vary with the severity of the condition to be alleviated. It is to be further understood that for any particular subject, specific dosage regimens should be adjusted over time according to the individual need and the professional judgement of the person administering or supervising the administration of the compositions. Examples of the techniques and protocols mentioned above can be found in Remington's Pharmaceutical Sciences, 16^{th} edition, Oslo, A. (ed), 1980.

Solutions or suspensions used for parenteral, intradermal, subcutaneous, or topical application may include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of toxicity such as sodium-chloride or dextrose.

Suitable pharmaceutically acceptable carriers for parenteral application, such as intravenous, subcutaneous, or intramuscular injection, include sterile water, physiological saline, bacteriostatic saline (saline containing 0.9 mg/ml benzyl alcohol) and phosphate-buffered saline. If administered intravenously, preferred carriers are physiological saline or phosphate buffered saline.

Methods for preparing transdermal formulations including topical formulations or transdermal delivery devices such as patches are known to those skilled in the art (see, for example, Brown and Langer, 1988).

Synthetic peptides of this invention may be prepared with carriers that will protect the synthetic peptide against rapid elimination from the body, such as through controlled release formulations, including implants and microencapsulated delivery systems. Biodegradable, biocompatible polymers can be used, such as ethylene vinyl acetate, polyanhydrides, polyglycolic acid, collagen, polyorthoesters, and polylactic acid.

Liposomal suspensions are also suitable carriers for the synthetic peptides of this invention. The synthetic peptides may be conjugated to a lipid by known methods for incorporation into a liposomal envelope or the compounds may be encapsulated into the liposome. Liposomes may be prepared according to methods known to those skilled in the art, such as is described in U.S. Pat. No. 4,522,811. For example, liposome formulations may be prepared by dissolving appropriate lipid(s) (such as stearoyl phosphatidyl ethanolamine stearoyl phosphatidyl choline, arachadoyl phosphatidy choline, and cholesterol) in an inorganic solvent that is then evaporated, leaving behind a thin film of dried lipid on the surface of the container. An aqueous solution of the active synthetic peptide of the invention or its monophosphate, and/or triphosphate derivatives are then introduced into the container. The container is then swirled by hand to free the lipid aggregates, thereby forming the liposomal suspension.

For nasal or pulmonary administration, the active ingredients will be in the form of a fine powder or a solution or suspension suitable for inhalation. Alternatively, the active ingredients may be in a form suitable for direct application to the nasal mucosa such as an ointment or cream, nasal spray, nasal drops or an aerosol.

Oral compositions may include an inert diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. Methods for encapsulating compositions (such as in a coating of hard gelatin) for oral administration are well known in the art (Baker and Richard, 1986). Techniques to overcome various barriers including the mucus-layer barrier, the enzymatic barrier, and the membrane barrier are well known in the art, (Bernkop-Schnurch et al, 2001).

Tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, or corn starch; a lubricant such as magnesium stearate; a glidant such as colloidal silicon dioxide; a sweetening agent such as sucrose or saccharin; or a flavouring agent such as peppermint, methyl salicylate, or orange flavouring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents. Alternatively, synthetic peptides of this invention could be administered as a component of an elixir, suspension, syrup, wafer, chewing gum or the like. A syrup may contain, in addition to the active synthetic peptides sucrose as a sweetening agent and certain preservatives, dyes and colourings and flavours.

The present invention is directed to a method of treating a myostatin related pathological condition in a mammal, wherein the method generally comprises at least the step of administering to a mammal in need thereof, an effective amount of at least one synthetic peptide of the invention having myostatin antagonistic activity, for a time sufficient to prevent, treat or ameliorate the symptoms of said myostatin related pathological condition. In preferred embodiments the mammal is a human that has, is suspected of having, or has been diagnosed with one or more myostatin related pathological conditions.

The pathologic condition is characterized, at least in part, by an abnormal amount, development or metabolic activity of muscle or adipose tissue in a mammal and may include disorders related to muscle hypertrophy; muscle atrophy and muscle wasting associated with inflammatory myopathies, muscular dystrophies, motor neuron diseases, diseases of the neuromuscular junction, diseases of the peripheral nerve, myopathies due to endocrine abnormalities, metabolic syndrome, HIV, cancer, sarcopenia, cachexia and other wasting conditions; cardiac failure; osteoporosis; renal failure or disease; liver failure or disease; anorexia; obesity; diabetes; and wound healing.

Inflammatory myopathies that can be treated include: Dermatomyositis (PM/DM), Inclusion Body Myositis (IBM) and Polymyositis (PM/DM).

Muscular dystrophies that can be treated include: Becker Muscular Dystrophy (BMD), Congenital Muscular Dystrophy (CMD), Distal Muscular Dystrophy (DD), Duchenne Muscular Dystrophy (DMD), Emery-Dreifuss Muscular Dystrophy (EDMD), Limb-Girdle Muscular Facioscapulohumeral Muscular Dystrophy (FSH or FSHD), Dystrophy (LGMID), Myotonic Dystrophy (MMD) and Oculopharyngeal Muscular Dystrophy (OPMD),

Motor neuron diseases that can be treated include: Adult Spinal Muscular Atrophy (SMA), Amyotrophic Lateral Sclerosis (ALS), Infantile Progressive Spinal Muscular Atrophy (SMA, SMA1 or WH), Intermediate Spinal Muscular Atrophy (SMA or SMA2), Juvenile Spinal Muscular Atrophy (SMA, SMA3 or KW) and Spinal Bulbar Muscular Atrophy (SBMA).

Diseases of the neuromuscular junction that can be treated include: Congenital Myasthenic Syndrome (CMS), Lambert-Eaton Syndrome (LES) and Myasthenia Gravis (MG).

Diseases of peripheral nerve that can be treated include: Charcot-Marie-Tooth Disease (CMT), Dejerine-Sottas Disease (DS), and Friedreich's Ataxia (FA).

Myopathies due to endocrine abnormalities that can be treated include: Hyperthyroid Myopathy (HYPTM) and Hypothyroid Myopathy (HYPOTM).

Other myopathies that can be treated include: Central Core Disease (CCD), Myotonia Congenita (MC), Nemaline Myopathy (NM), Myotubular Myopathy (MTM or MM), Paramyotonia Congenita (PC) and Periodic Paralysis (PP).

Metabolic diseases of muscle that can be treated include: Acid Maltase Deficiency (AMD), Carnitine Deficiency (CD), Carnitine Palmityl Transferase Deficiency (CPT), Debrancher Enzyme Deficiency (DBD), diabetes, Lactate Dehydrogenase Deficiency (LDHA), Myoadenylate Deaminase Deficiency (MAD) Mitochondrial Myopathy (MITO), obesity Phosphorylase Deficiency (MPD or PYGM), Phosphofructokinase Deficiency (PFKM), Phosphoglycerate Kinase Deficiency (PGK) and Phosphoglycerate Mutase Deficiency (PGAM or PGAMM).

The synthetic peptide of the invention can also be used for treating or preventing congestive heart failure; for reducing frailty associated with aging; for increasing bone density (such as for treating osteoporosis) or accelerating bone fracture repair; for treating growth retardation, for the treatment of physiological short stature, for attenuating protein catabolic response such as after a major operation; for reducing protein loss due to chronic illness; for accelerating wound healing; for accelerating the recovery of burn patients or patients having undergone major surgery; for maintenance of skin thickness; for maintaining metabolic homeostasis, for treating renal failure/disease and liver failure/disease; for treating growth hormone deficient adults and for preventing catabolic side effects of glucocorticoids; and for treating a number of neuronal system disease conditions, including CNS injuries/disease such as spinal cord injury and stroke, and PNS injuries/diseases.

These disorders can be treated by administering a therapeutic amount of one or more synthetic myostatin antagonist peptides to a subject in need thereof.

In a further embodiment, the invention contemplates the use of one or more muscle growth factors which may be co-administered with the pharmaceutical compositions of the present invention to give an additive or synergistic effect to the treatment regime. Such growth factors may be selected from the group consisting of HGF, FGF, IGF, MGF, growth hormone etc. Such growth factors may be administered either separately, sequentially or simultaneously with the pharmaceutical compositions comprising at least one polypeptide of the invention having myostatin antagonist activity.

In a further embodiment, the invention contemplates the use of a second pharmacologically active compound having different activity to the synthetic myostatin antagonist peptides of the invention to be used conjointly with the synthetic peptide of the invention to treat the myostatin related disorders. For example, the synthetic peptide may be administered in conjunction with active compounds selected from the group consisting of polypeptide growth factors (as mentioned above), NSAIDs or COX-2 inhibitors, alpha and beta blockers, ACE inhibitors, bisphosphonates, oestrogen receptor modulators, antihypertensive agents, glutamate antagonists, insulin, antibiotics, protein kinase C inhibitors or various over the counter substances as would be appreciated by a skilled worker. Such active compounds may be administered either separately, sequentially or simultaneously with the at least one synthetic peptide of the invention having myostatin antagonist activity.

The present invention is also directed to the use of one or more synthetic peptides of the invention in the manufacture of a medicament for treating myostatin related pathological conditions in a patient in need thereof.

The medicament may be formulated for local or systemic administration. For example, the medicament may be formulated for injection directly into a muscle, or may be formulated for oral administration for systemic delivery to the muscle for the treatment of muscle wasting conditions. The medicament may be formulated for topical administration for the treatment of wound healing, and may be formulated for oral administration for the treatment of obesity and diabetes.

The medicament may further comprise one or more additional muscle growth promoting compounds to give an additive or synergistic effect to the treatment of muscle wasting conditions or for increasing muscle mass. The medicament may be formulated for separate, sequential or simultaneous administration of the one or more synthetic peptides of the invention and the one or more muscle growth promoting compounds.

Without being bound by theory, it is thought that the novel synthetic peptides of the present invention, which have myostatin antagonistic activity, will be effective at preventing or treating myostatin related disorders in part via three mechanisms. Firstly by inducing satellite cell activation, proliferation and differentiation. Satellite cells are the muscle stem cells and are thus involved in muscle tissue regeneration. Secondly, by enhancing myoblast proliferation and migration to the site of regeneration, and thirdly by enhancing macrophage recruitment. It is known that macrophages act to attract myoblasts and thus increase myogenesis. The effect on macrophage recruitment has previously been observed with other myostatin antagonists to result in improved wound healing (WO2006/083182 and WO 2008/016314). Thus, the present invention should also be effective at improving wound healing.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Synthetic Myostatin Antagonist Peptides Increase Myoblast Proliferation In Vitro

### Methods

### Expression and Purification of Recombinant Control Myostatin Antagonist Peptide

A cDNA corresponding to the 267-310 amino acids of bovine myostatin sequence, hereafter referred to as myostatin antagonist 310, was individually PCR amplified and cloned into a pET16-B vector using BamHI sites. Expression and purification of myostatin antagonists 310 was done according to the manufacturer's (Qiagen) protocol under native conditions yielding peptides with an N terminal (M G H H H H H H H H H H S S G H I E G R H M L E D P) and C terminal tag (E D P A A N K A R K E A E L A A A T A E Q). This recombinant peptide was the positive control to compare activity with the synthetic versions.

### Production of Synthetic Myostatin Antagnost Peptide

Synthetic peptides corresponding to amino acids 267-310 of the bovine myostatin sequence, hereinafter referred to as the 310 synthetic peptide were produced, either having a 2-3 connectivity or having a 2-3/1-4 connectivity by Bachem Americas, Inc, CA, US. These peptides were synthesized by coupling the carboxyl group or C-terminus of one amino acid to the amino group or N-terminus of another. Protecting groups were used to prevent unwanted cystein bonding.

Specifically, the peptides of the present invention were made using solid phase techniques, whereby peptide chains are built on small solid polymer beads using repeated cycles of coupling-deprotection. Once the desired peptide was made the bond between the first amino acid and the polymer was broken to give the free peptide. At this stage the side-chain protecting groups were also removed. After this step the crude peptide was purified by reversed-phase preparative high performance liquid chromatography and finally lyophilized.

Selective disulfide bond formation and/or prevention was achieved by applying complex orthogonal protection schemes in order to get well defined conformations as would be understood by a skilled worker and as set out in the Bachem Peptide Users Guide, July 2009, Bachem AG Switzerland.

The composition of a peptide was confirmed by amino acid analysis or sequencing.

### Myoblast Assay

Bovine C2C12 myoblasts were grown in Dulbecco's modified eagle medium according to the standard techniques (Thomas et al, 2000). The myoblast proliferation assay was carried out in uncoated 96-well microtitre plates. C2C12 cultures were seeded at 1000 cells/well. Following a 16-hour attachment period, test media containing varying concentrations of either the synthetic 310 peptide in the 2-3 conformation, the synthetic 310 peptide in the 1-4/2-3 conformation or the positive control (recombinant 310 peptide) were added and cells were incubated for a further 48 or 72 hour period. After the incubation period, proliferation was assessed using methylene blue photometric endpoint assay as previously described (Thomas et al, 2000).

### Results

The results show that the 310 recombinant positive control peptide significantly enhanced myoblast proliferation over 48 or 72 hours compared to control (media and buffer only) at a concentration of 10 and 20 μg. Both of the synthetic 310 peptides also enhanced the proliferation capacity of myoblasts.

The 310 synthetic peptides were not as effective as the recombinant 310 control peptide, but still resulted in a significant increase in myoblast proliferation demonstrating that the synthetic 310 peptides can effectively accelerate muscle regeneration by enhancing myoblast proliferation.

The 310 synthetic peptide having the 2-3 connectivity only was biologically active and the 310 synthetic peptide with the 1-4/2-3 connectivity showed slightly more biological activity. It is likely that the biological activity of this peptide is due to the presence of the 2-3 connectivity and the addition of the 1-4 connectivity may have added to this core activity by its 3-D structure, for example.

The present inventors postulate that the 2-3 connectivity in the synthetic mature myostatin peptides of the present invention is responsible for and therefore crucial to, bioactivity.

The results are shown in Table 1, below:

TABLE 1
1. Synthetic 310 (2-3/1-4 connectivity)

| | % proliferation above control* | |
|---|---|---|
| | 48 h | 72 h |
| Media - buffer (control) | 100.00 | 100.00 |
| 310 positive control (10 μg) | 111.76 | 119.64 |
| Synthetic 310 (0.1 μg) | 100.17 | 100.54 |
| Synthetic 310 (1.0 μg) | 100.07 | 102.60 |
| Synthetic 310 (10.0 μg) | 108.58 | 107.51 |
| Synthetk 310 (20.0 μg) | 106.87 | 110.47 |
| 2. Synthetic 310 (2-3 connectivity) | | |
| Media - buffer (control) | 100.00 | 100.00 |
| 310 positive control (10 μg) | 113.58 | 122.97 |
| Synthetic 310 (0.1 μg) | 101.66 | 101.06 |
| Synthetic 310 (1.0 μg) | 97.70 | 102.00 |
| Synthetic 310 (10.0 μg) | 104.18 | 103.83 |
| Synthetic 310 (20.0 μg) | 105.00 | 105.07 |

*absorbance at 655 OD is correlated at 100% for the control (media + buffer only) and the test absorbance calibrated to this.

### Example 2

### In Vitro Myoblast Proliferation by Synthetic Myostatin Peptides

To confirm that the increased proliferation observed in example 1 was not simply false positive results, the plates were used to count nuclei.

### Methods

Cells in the wells of interest were stained with DAPI (4′-6-diamidino-2-phenylindole) which is a fluorescent dye that stains nuclei blue. DAPI was used at a concentration of 1 g/ml in PBS and cells stained for 5 mins at room temperature, then washed once with PBS buffer. Images of each well were then captured via a Leica DMI 6000 microscope and an image analysis program (Image-Pro plus) utilised to count the nuclei. For each treatment (recombinant 310 positive control, and synthetic peptides (2-3) and (1-4/2-3) at 10 and 20 g/ml), 3-6 wells per treatment were analysed.

### Results

There were significant increases in cell numbers at 48 h and 72 h (as shown in Table 2, below) for the synthetic 310 (2-3) peptide and at 72 h for the synthetic 310 (1-4/2-3) peptide. These results confirm that the increases in myoblast proliferation observed in example 1 was a result of a genuine increase in cell numbers.

TABLE 2
1. Synthetic 310 (2-3 connectivity)

| | Number of nuclei per well | |
|---|---|---|
| | 48 h | 72 h |
| Media - buffer (control) | 707.7 | 1859.1 |
| 310 positive control (10 μg) | 816.7 | 2229.2 |
| Synthetic 310 (10.0 μg) | 810.5 | 1906.1 |
| Synthetic 310 (20.0 μg) | 808.9 | 1903.6 |

Synthetic 310 (1-4/2-3 connectivity)

| | | |
|---|---|---|
| Media - buffer (control) | 799.8 | 1986.9 |
| 310 positive control (10 μg) | 847.1 | 2429.3 |
| Synthetic 310 (10.0 μg) | 810.2 | 2328.2 |
| Synthetic 310 (20.0 μg) | 739.3 | 2172.0 |

### Example 3

### Synthetic Myostatin Antagonist Peptides that Lack Biological Activity

### Methods

A number of synthetic peptides were prepared having an amino acid sequence corresponding to amino acids at positions 267-310 and 282-310 of SEQ ID NO:1. Of the 267-310 peptides a number of different connectivities were prepared as follows:
1. 1-3/2-4
2. 1-2/3-4
3. 1-2
4. 1-3
5. 1-4
6. 1-2, 3-4/1-3, 2-4
7. 2-4
8. 3-4
9. 1-4/2-3
10. Linear (no forced connectivity)

The peptides were synthesized as described above in example 1. Different cysteine residues were protected and deprotected to produce the specified connectivities and would be understood by a skilled worker.

### Results

The only synthetic peptide tested that showed enhanced myoblast proliferation in vivo was the linear 310 synthetic peptide. None of the other 310 conformations showed any activity. The 310 synthetic peptide that was N-terminally truncated at amino acid position 282 was also not active. This particular peptide only included two cysteine residues, namely cysteine 3 and 4 so would comprise the 3-4 connectivity. The linear 310 synthetic peptide would have folded into any one or more of the possible confirmations. As this molecule was effective at enhancing myoblast proliferation in vitro, the inventors summise that it included the 2-3 connectivity which appears to be the bioactive confirmation (Results not shown).

### CONCLUSION

Synthetic myostatin antagonist peptides that include at least two cysteine residues, whereby cysteine residues at positions 281 and 282 of SEQ ID NO: 1 bond to form a disulfide bond (corresponding to the 2-3 connectivity) are effective at enhancing myoblast proliferation in vitro. We show synthetic peptides that have either 2-3 or 2-3/1-4 connectivities have myostatin antagonist activity.

### REFERENCES

The following references, to the extent that they provide exemplary, procedural or other details supplementary to those set forth herein, are specifically incorporated herein by reference. The patent specifications, referred to throughout the text of this specification, are also specifically incorporated herein by reference.
Altschul et al, Nucleic Acid Res. 25: 3389 (1997)
Baker, R, Controlled Release of Biologically Active Agents, John Wiley & Sons, 1986
Bernkop-Schnurch A and Walker G (2001) Multifunctional matrices for oral peptide delivery. Crit Rev Ther Drug Carrier Syst 18(5): 459-501
Brown & Langer Transderman Reliving of Drugs (1998). Annual Review of Medicine, 39: 221-229
Carlson, C J, Booth, F W and Gordon, S E. (1999). Skeletal muscle myostatin mRNA expression is fibre-type specific and increases during hind-limb unloading. Am J Physiol 277, R601-6
Giridharan N V (1998). Animal models of obesity and their usefulness in molecular approach to obesity. Indian J Med Res 108, 225-42.
Gonzalez-Cadavid N F, Taylor W E, Yarasheski K, Sinha-Hikim I, Ma K, Ezzat S, Shen R, Lalani R, Asa S, Mamita M, Nair G, Arver S, Bhasin S. (1998) Organization of the human myostatin gene and expression in healthy men and HIV-infected men with muscle wasting. Proc Natl Acad Sci USA 95: 14938-43.
Jeanplong F, Sharma M, Somers W G, Bass J J and Kambadur R (2001) Genomic organization and neonatal expression of the bovine myostatin gene. Mol Cell Biochem 220:31-37.
Kirk S, Oldham J, Kambadur R, Sharma M, Dobbie P, and Bass J. (2000). Myostatin regulation during skeletal muscle regeneration. J Cell Physiol 184(3), 356-63.
Langley B, Thomas M, McFarlane C, Gilmour S, Sharma M, Kambadur R. (2004) Myostatin inhibits rhabdomyosarcoma cell proliferation through an Rb-independent pathway. Oncogene 23: 524-34.
Lee S J, McPherron A C. (2001) Regulation of myostatin activity and muscle growth. Proc Natl Acad Sci USA 98: 9306-9311.
Like A A, Rossini A A (1976). Streptozotocin-induced pancreatic insulitis: new model of diabetes mellitus. Science 193, 415-7.
Ma K, Mallidis C, Bhasin S, Mahabadi V, Artaza J, Gonzalez-Cadavid N, Arias J, Salehian B. (2003) Glucocorticoid-induced skeletal muscle atrophy is associated with upregulation of myostatin gene expression. Am J Physiol Endocrinol Metab 285: E363-E371.
McCroskery S, Thomas M, Maxwell L, Sharma M, and Kambadur R. (2003). Myostatin negatively regulates satellite cell activation and self-renewal. J Cel Biol 162.
Shukla et al (1998), Differential protein expression during healing of cutaneous wounds in experimental normal and chronic models. Biochem Biophys Res Commun 244(2), 434-9.
Pearson W R and Lipman D J (1988). Improved tools for biological sequence comparison. Proc Natl Acad Sci USA 85(8), 2444-8.
Tanabe Y, Esaki K, Nomura T (1986). Skeletal muscle pathology in X chromosome-linked muscular dystrophy (mdx) mouse. Acta Neuropathol (Berl) 69(1-2), 91-5.
Thomas M, Langley B, Berry C, et al. (2000) Myostatin, a negative regulator of muscle growth, functions by inhibiting myoblast proliferation. J Biol Chem 275: 40235-40243.
Wehling M, Cai B, and Tidball J G (2000). Modulation of myostatin expression during modified muscle use. Faseb J 14, 103-10.
Werle M. and Bernkop-Schnurch A. (2006) Strategies to improve plasma half life of peptide and protein drugs. Amino Acids 30: 351-367.
Yang L, Scott P G, Dodd C, Medina A, Jiao H, Shankowsky H A, Ghahry A and Tredget E E. (2005). Identification of fibrocytes in postburn hypertrophic scar. Wound Repair and Regeneration 13(4), 398-404.

### INDUSTRIAL APPLICATION

The present invention provides novel synthetic peptides having myostatin antagonistic activity which will be useful in the treatment of myostatin related disorders. Such disorders are characterised, at least in part, by an abnormal amount, development or metabolic activity of muscle or adipose tissue in a mammal and may include disorders related to muscle hypertrophy; muscle atrophy and muscle wasting associated with inflammatory myopathies, muscular dystrophies, motor neuron diseases, diseases of the neuromuscular junction, diseases of the peripheral nerve, myopathies due to endocrine abnormalities, metabolic syndrome, HIV, cancer, sarcopenia, cachexia and other wasting conditions; cardiac failure; osteoporosis; renal failure or disease; liver failure or disease; anorexia; obesity; diabetes; and wound healing.

1. A synthetic peptide having myostatin antagonist activity having an amino acid sequence corresponding to at least five contiguous amino acids of a mature myostatin peptide of SEQ ID NO:1, wherein the synthetic peptide comprises at least two cysteine residues at positions 281 and 282 which are forced to bond and form a disulfide bond, or a functional variant or fragment thereof.

2. The synthetic peptide of claim 1, having at least ten, at least fifteen, at least twenty, at least twenty five, at least thirty, at least thirty five, at least forty, at least forty five, at least fifty, at least fifty five, or at least sixty contiguous amino acids of SEQ ID NO: 1.

3. The synthetic peptide of claim 1, comprising an amino acid sequence corresponding to a C-terminally truncated mature myostatin peptide of SEQ ID NO: 1, wherein the C-terminal truncation is at a position at or between amino acids 282 and 335.

4. The synthetic peptide of claim 3, wherein the C-terminal truncation is at the amino acid position selected from the group consisting of 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334 and 335 or a functional variant or fragment thereof.

5. The synthetic peptide of claim 4, wherein the C-terminal truncation is at the amino acid position selected from the group consisting of 329, 320, 310, 300, 295, 289, 284 or 282 (SEQ ID NOS: 2-9 respectively), or a functional variant or fragment thereof, or a polypeptide having substantial sequence homology thereto.

6. The synthetic peptide of claim 5, consisting of a C-terminally truncated mature myostatin polypeptide, wherein the C-terminal truncation is at amino acid position 310 (SEQ ID NO: 4).

7. The synthetic peptide of claim 3, further comprising a N-terminal truncation at or between amino acid position 268 to 280.

8. A pharmaceutical composition comprising the synthetic peptide of claim 1 together with a pharmaceutically acceptable carrier.

9. A method of regulating muscle growth, promoting adipogenic differentiation and/or promoting bone growth or mineralization in an animal in need thereof, said method comprising administering to said animal an effective amount of the synthetic peptide of claim 1.

10. The method as of claim 9 for producing increased muscle mass, decreased fat deposition and/or improved bone growth in a sheep, cattle, deer, poultry, turkey, pig, horse, mouse, rat, cat, dog or human.

11. A method to prevent, treat or reduce the severity of a myostatin related pathologic condition in a patient, wherein said condition is characterized, at least in part, by an abnormal amount, development or metabolic activity of muscle or adipose tissue, wherein said method comprises administering an effective amount of the synthetic peptide of claim 1 to a patient in need thereof.

12. The method of claim 11, wherein the pathologic condition is selected from the group consisting of disorders related to muscle hypertrophy; muscle atrophy and muscle wasting associated with inflammation myopathies, muscular dystrophies, motor neuron diseases, diseases of the neuromuscular junction, diseases of the peripheral nerve, myopathies due to endocrine abnormalities, metabolic syndrome, HIV, cancer, sarcopenia, cachexia and other wasting conditions; cardiac failure; osteoporosis; renal failure or disease; liver failure or disease; anorexia; obesity; diabetes; and wound healing.

13. A method to prevent, treat or ameliorate a condition wherein the condition is one that would benefit, in part, from increased satellite cell activation, myoblast proliferation, macrophage and myoblast migration and/or reduced fibrosis wherein said method comprises administering an effective amount of the synthetic peptide of claim 1 to a patient in need thereof.

14. The method of claim 13 wherein the condition is selected from the group consisting of muscle damage due to trauma; muscle damage due to the administration of agents such as chemotherapy agents, radiation therapy, desamethasone; muscle wasting due to prolonged bed rest such as that required after surgery; wound healing; disorders related to muscle hypertrophy; muscle atrophy and muscle damage associated with inflammatory myopathies, muscular dystrophies, motor neuron diseases, diseases of the neuromuscular junction, diseases of the peripheral nerve, myopathies due to endocrine abnormalities, metabolic syndrome, HIV, cancer, sarcopenia, cachexia and other wasting conditions; cardiac failure; osteoporosis; renal failure or disease; liver failure or disease; anorexia; obesity and diabetes.

15. The pharmaceutical composition of claim 8, wherein the synthetic peptide is conjugated to a second pharmaceutically active compound to enhance the therapeutic effect on the target cell or tissue, and wherein the pharmaceutical composition is formulated for separate, sequential or simultaneous administration of the synthetic peptide and the second compound.

16. A method of regulating muscle growth of an animal comprising administering to said animal an effective amount of the synthetic peptide of claim 1.

17. The method of claim 16, to produce increased muscle mass in a sheep, cattle, deer, poultry, turkey, pig, horse, cat, dog or human.

18.-23. (canceled)

24. A method of producing the synthetic peptide of claim 1, said method comprising the steps:
a. preparing a peptide chain having an amino acid sequence corresponding to at least five contiguous amino acids of a mature myostatin peptide of SEQ ID NO: 1, wherein the peptide chain includes at least the two cysteine residues at positions 281 and 282, using solid phase peptide synthesis;
b. protecting and deprotecting the two cysteine residues at positions 281 and 282 to ensure that the two cysteine residues at positions 281 and 282 bond to form a disulfide bond.

25. The method of claim 24, wherein when the synthetic peptide comprises cysteine residues at positions 272, 281, 282 and 309, the protecting and deprotecting step b ensures that the cysteine residues at positions 281 and 282 only bond to form a disulfide bond, or that the cysteine residues at positions 281 and 282 bond and the cysteine residues at positions 272 and 309 bond, to form disulfide bonds.

26. A synthetic peptide produced by the method of claim 24.


SEQUENCE LISTING (9 sequences)

SEQ ID NO: 1 - protein, 375 aa, Homo sapiens
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 2 - protein, 63 aa, Artificial Sequence
  Myostatin Polypeptide Fragments
DFGLDCDEHSTESRCCRYPLTVDFEAFGWDWIIAPKRYKANYCSGECEFVFLQKYPHTHLVHQ

SEQ ID NO: 3 - protein, 54 aa, Artificial Sequence
  Myostatin Polypeptide Fragments
DFGLDCDEHSTESRCCRYPLTVDFEAFGWDWIIAPKRYKANYCSGECEFVFLQK

SEQ ID NO: 4 - protein, 44 aa, Artificial Sequence
  Myostatin Polypeptide Fragments
DFGLDCDEHSTESRCCRYPLTVDFEAFGWDWIIAPKRYKANYCS

SEQ ID NO: 5 - protein, 34 aa, Artificial Sequence
  Myostatin Polypeptide Fragments
DFGLDCDEHSTESRCCRYPLTVDFEAFGWDWIIA

SEQ ID NO: 6 - protein, 28 aa, Artificial Sequence
  Myostatin Polypeptide Fragments
DFGLDCDEHSTESRCCRYPLTVDFEAFG

SEQ ID NO: 7 - protein, 23 aa, Artificial Sequence
  Myostatin Polypeptide Fragments
DFGLDCDEHSTESRCCRYPLTVD

SEQ ID NO: 8 - protein, 18 aa, Artificial Sequence
  Myostatin Polypeptide Fragments
DFGLDCDEHSTESRCCRY

SEQ ID NO: 9 - protein, 16 aa, Artificial Sequence
  Myostatin Polypeptide Fragments
DFGLDCDEHSTESRCC